(19) United States
(12) Patent Application Publication
(10) Pub. No.: US 2009/0028830 A1
(43) Pub. Date: Jan. 29, 2009

(54) Preparation of multipotent stem cells and the use thereof

(76) Inventors: Yung-Hsiang Liu, Taipei (TW); Wing-Yee Chan, Taipei (TW); Yuan-Feng Lin, Taipei (TW)

Correspondence Address: TROXELL LAW OFFICE PLLC;SUITE 1404, 5205 LEESBURG PIKE, FALLS CHURCH, VA, 22041, US

(21) Appl. No.: 11/445,320

(22) Filed: Jun. 2, 2006

(30) Foreign Application Priority Data
Jun. 2, 2005 (TW) 094118270

(51) Int. Cl. A61K 35/12; C12N 5/00; A61P 43/00

(52) U.S. Cl. 424/93.7; 435/325; 435/377

(57) ABSTRACT

The invention relates to a method for the enriched induction of multipotent stem cells, named P-stem cells, from CD14⁺ peripheral monocytic cells. P-stem cells are capable of differentiating into osteoblasts, chondrocytes, neuron cells, etc. Also disclosed relates to a method for tissue repairing by in vivo implanting P-stem cells into damaged tissues.

### BACKGROUND OF THE INVENTION

The invention relates to a method for the enriched induction of multipotent stem cells, named P-stem cells, from CD14⁺ peripheral monocytic cells. P-stem cells are capable of differentiating into osteoblasts, chondrocytes, neuron cells, etc. Also disclosed relates to a method for tissue repairing by in vivo implanting P-stem cells into damaged tissues.

Recently, stem cells used in clinical cell therapies were mainly collected from bone marrow and core blood. However, the content of those stem cells in whole blood cells is rare and allogenic transplantation of which usually generates the host immune rejection.

CD34⁺ hematopoitic stem cells are isolated by CD34 antibody from bone marrow and extensively applied for cell therapies in current clinic. However, the rare proportion of CD34⁺ hematopoitic stem cells in whole blood cells (˜1/100,000) and the allogenic rejection of transplantation lead to the limitation of their application in the clinic. Although researches in this field were attempting to develop the method to efficiently manipulate the proliferation of CD34⁺ hematopoitic stem cells in the in vitro cultivation, the allogenic rejection of transplantation is still major issue and disadvantage of their application in the clinic.

Since 1990, the isolation of stem cells from core blood was become to be other way than bone marrow. The amount of stem cells isolated from core blood is more than that of bone marrow. In addition, the differentiating capacity of core blood-derived stem cells is greater than bone marrow-derived stem cells. Therefore, Core Blood Bank was widely developed on the earth to preserve core blood from newborns and offer the future stem cell-related researches and clinical cell therapies. Although the amount of stem cells isolated from core blood is more than that of bone marrow, the in vitro proliferation of core blood-derived stem cells is still needed to obtain the sufficient amount for clinical cell therapies. Furthermore, the particular preservation of core blood in low temperature is very expensive, and blood cells intend to death when the temperature of preservation is unstable. Besides, the histocompatibility is another critical issue for the clinical cell therapy of core blood-derived stem cells. This issue not only increases the cost of the therapy but also decline the successful rate of transplantation.

Several issues are concerned in the clinical cell therapies using bone marrow- and core blood-derived stem cells. 1) The proportion of bone marrow- and core blood-derived stem cells in whole blood are rare. Their in vitro cultivation is needed to obtain the sufficient amount for clinical cell therapies. However, the efficient induction of their proliferation is still not reported. 2) The long-term preservation of core blood at low temperature was promised to be safety by Core Blood Bank. However, the cell viability after thawing is still needed to further estimation. 3) The donor is susceptible to the pain and anesthetic risk during the bone marrow puncture to collect stem cells. 4) Rejection: The immunity of recipient might reject the transplanted stem cells, which leads to decline in the efficiency of transplantation. 5) The core blood collection is once a life for everyone.

### SUMMARY OF THE INVENTION

The inventor offers a method to prepare autologous stem cells and provide a feasibility of those cells in clinical application.

The first object of this invention is to provide P-stem cells. The fundamental is that one of monocytic cell population treated with at least one of protein kinase C (PKC) modulator to directly differentiate the monocytic cells towards multipotent P-stem cells.

The second object of this invention is to offer a target cells. The fundamental is that P-stem cells treated with at least one of differentiation factors to induce their differentiation into target cells, such as chondrocytes, osteoblast, neuron cells, etc.

The third object of this invention is to offer a P-stem cell-based repairing agent. The fundamental is that the P-stem cell-based repairing agent transplants to the lesion where P-stem cells differentiate to become the target cell which consequently repair the lesion.

The fourth object of this invention is to offer a target cell-based repairing agent. The fundamental is to directly transplant at least one of the target cells to repair the lesion.

The invention is to fully differentiate mononucleated cells, such as peripheral monocytes, into multipotent P-stem cells. The proportion of monocytes, one of so-called mononucleated cells, is about 10% of total leukocytes. In the physiological condition, one milliliter of peripheral blood contains 5,000 to 10,000 leukocytes or 500 to 1,000 monocytes at least. Furthermore, the 100 ml of peripheral bloods should contain 50,000 to 100,000 monocytes. The employment of this invention can promptly induce the differentiation of those 50,000 to 100,000 monocytes to become P-stem cells.

The first advance of this invention is to differentiate peripheral monocytes towards P-stem cells. The amount of peripheral monocyte-derived P-stem cells is more than (1,000 to 10,000 folds) bone marrow- and core blood-derived stem cells. The second advance of this invention is that the autologous transplantation of P-stem cells dose not concern the immune rejection.

The reproducibility of P-stem cell differentiation from peripheral monocytes and the convenience of peripheral blood collection from veins are great advance of this invention. Unlike collecting stem cells from bone marrow, the donor must take a risk in the process of bone marrow puncture. The collection of peripheral blood collection can be repeatable, but core blood collection is once a life of man. The particular preservation (−180° C. liquid nitrogen) of bone marrow- and core blood-derived stem cells cause rise in the cost of therapy, which may, in turn, elevate the difficulty in their clinical application.

This invention regarding P-stem cells is capable of differentiating into target cells, such as chondrocytes, osteoblasts, neuron cells, etc, suggests that P-stem cells, similar to bone marrow- and core blood-derived stem cells, is a multipotent progenitor cells. Those P-stem cell-derived target cells are able to directly repair the damaged tissues. For example, the transplantation of P-stem cell-derived chondrocytes into damaged joints might promptly replenish the amount of chondrocytes and repair the damaged joints. Furthermore, the transplantation of P-stem cell-derived neuron cells into the lesions might also provide efficient repair of damaged neurons.

In this invention, P-stem cells are able to differentiate towards many cell (tissue) types of human, such as hepatocytes, brain cells, neuron cells, cliondrocytes, adipocytes, ophthalmic tissue, acoustic tissue, pancreatic tissue, cardiocytes, myocytes, keratinocytes, osteoblasts. bile tissue, vascular tissue, renal tissue, bone marrow tissue, pulmonary tissue, follicular tissue, gastric-intestine tissue, digestion tissue, reproductive tissue, etc. Moreover, the autologous transplantation of P-stem cell-derived cells (tissues) into recipients does not induce an immune rejection.

This invention also provides a method of P-stem cell-derived cell (tissue)-dependent tissue repairing to directly repair and reconstruct the damaged tissue. For example, the cardiac tissue damage of Patient A can be repaired by the autologous transplantation of Patient A's P-stem cells into the damaged tissue where P-stem cells can promptly differentiate into cardiocytes. The cardiac failure will be readily recovered after the P-stem cell-derived cardiocytes replenishing the lost of original cardiocytes. The ideal has been previously carried out by transplanting bone marrow- or core blood-derived stem cells into damaged cardiac tissue. In accord with previous reports in the transplantation of bone marrow- or core blood-derived stem cells, transplanting P-stem cells into bone marrow can improve the hematopoiesis of leukemia and transplanting P-stem cells into cardiac tissue can treat myocardial infarction. The ideal is also feasible to treat hepatic and renal failures. P-stem cells can be used to recover any tissue damages of patients. These tissues includes hepatocytes, brain cells, neuron cells, chondrocytes, adipocytes, ophthalmic tissue, acoustic tissue, pancreatic tissue, cardiocytes, myocytes, keratinocytes, osteoblasts, bile tissue, vascular tissue, renal tissue, bone marrow tissue, pulmonary tissue, follicular tissue, gastric-intestinal tissue, digestion tissue, reproductive tissue, etc.

### DETAILED DESCRIPTION OF PREFERRED EMBODIMENTS

### Preparation of P-Stem Cells

Human peripheral blood (20 ml) is collected in the tube or syringe containing heparin, an anticoagulant. The mononuclear blood cells, such as monocytes are isolated by Flow-Cytometry using fluorescein-conjugated CD14 antibody and then cultured in RPMI-1640 containing 10% fetal bovine serum.

### PRACTICE EXAMPLE 1-1

Protein kinase C (PKC) inhibitors, Go6976 for example, add to culture medium at a range of concentration 0.1 to 10 μM. Mononucleated cells are incubated with Go6976 for 30 minutes at 37° C. PKC activators, Bryostatin-1 for example, then add to the culture at a range of concentration 1 to 100 nM. Cell culture is performed at 37° C. with 5% CO₂ for 15 to 21 days. Mononucleated cells will be fully differentiated into P-stem cells.

### PRACTICE EXAMPLE 1-2

Mononucleated cells are treated with granulocyte/macropbzage colony-stimulating factor (GM-CSF) (1.00 to 1,000 IU/ml) and stromal cell-derived factor (SDF-1) (10 to 100 nM) for 3 to 7 days at 37° C. with 5% CO₂. Mononucleated cells will be fully differentiated into P-stem cells.

### PRACTICE EXAMPLE 1-3

Mononuclcated cells are seeded on collagen- or fibronectin-precoated culture plate and cultured in RPMI-1640 medium containing 10% fetal bovine serum for 7 to 14 days with 5% CO₂. Mononucleated cells will be fully differentiated into P-stem cells.

The magnetic particle-conjugated CD14 antibody is one of methods to isolate mononucleated cells from peripheral blood (See FIG. 1). The mononucleated cells are not limited in peripheral blood cells. For tissue repairing, P-stem cells can be resuspended in normal saline (0.85% NaCl) and then transplanted into damaged tissues. In the above Practice Examples, PKC modulator is not limited to be Go6976, Bryostatin-1, GM-CSF, SDF-1, collagen, or fibronectin. Substances modulating PKC activity are capable of inducing the generation of P-stem cells from their progenitor cells.

### PRACTICE EXAMPLE 2

P-stem cells are identified as CD14 positive cells by Flow-Cytometry analysis with fluorescein-conjugated CD14 antibody. Briefly, P-stem cell suspension (0.5 ml) is incubated with 10 μl of fluorescein-conjugated CD14 antibody for 30 minutes at 4° C. After the incubation. P-stem cells are centrifuged at 1,000 rpm for 10 minutes, washed with normal saline for 3 times, and then analyzed by Flow-Cytometry.

### PRACTICE EXAMPLE 3

P-stem cells are cultured in osteogenic medium [low-glucose DMEM (Dulbecco's Modified Eagle Medium) containing osteogenic differentiating factor, such as 100 nM of dexamethasone, 10 mM of β-glycerophosphate, or 100 μg/ml of ascorbic acid.] for 14 days at 37° C. with 5% CO2. P-stem cells can fully differentiate into osteoblasts.

### PRACTICE EXAMPLE 4

The identification of P-stem cell-derived osteoblasts are usually performed by staining intracellular calcium deposition with alizarin red and determining intracellular alkaline phosphatase activity. FIG. 2A shows intracellular calcium deposition of P-stem cell-derived osteobtasts (red area, 200× magnification). FIG. 2B shows the intracellular alkaline phosphatase activity of P-stem cell-derived osteoblasts. Briefly, equal amount of P-stem cells and P-stem cell-derived osteoblasts are lyzed in equal volume of lysis buffer. Subsequently, 1-ml cell lysate of P-stem cells or P-stem cell-derived osteoblasts is incubated with 0.3 ml of alkaline phosphatase substrate, p-nitrophenyl phosphateis (pNPP), for 15 minutes. The yellow product generated by the reaction of alkaline phosphatase and pNPP is read at 405 nm by spectrophotometer. As shown in FIG. 2B, the intracellular alkaline phosphatase activity is 6-fold higher than that of P-stem cells (FIG. 2B).

### PRACTICE EXAMPLE 5

P-stem cells are cultured in chondrogenic medium [low-glucose DMEM containing chondrogenic differentiating factor, such as 100 nM of dexamethasone or 10 ng/ml of Transforming growth factor-betal (TGF-β1)] for 21 days at 37° C. with 5% CO₂. P-stem cells can fully differentiate into chondrocytes.

### PRACTICE EXAMPLE 6

FIG. 2C shows the microscopic observation of chondrocytes. The cultured chondrocytes exhibit a polygonal cell type. Safranin O staining is usually used to stain intracellular mucin of chondrocytes (FIG. 2D, red area).

### PRACTICE EXAMPLE 7

P-stem cells are cultured in neurogenic medium [α-minimum essential medium. (α-MEM) containing neurogenic differentiating factor, such as 50 μM Mercaptoethanol, 1 μM retinoic acid, 0.5 mM L-glutamine, 1% N2 supplement, and 2% B27 supplement] for 14 days at 37° C. with 5% CO₂. P-stein cells can fully differentiate into neuron cells.

### PRACTICE EXAMPLE 8

The immunostaining of glutaminic acid decarboxylase (GAD) and nestin is used to identify the generation of neuron cells. FIGS. 2F, and 2F shows that GAD and nestin are expressed in the cytoplasm of P-stem cell-derived neuron cells.

Besides, P-stem cells can differentiate into skeletal, myocyte, cardiomyocyte, renal cell, pulmonary cell, hepatocyte, and adipocyte in the conditioned media. For example:

1) culturing P-stem cells in skeletal myogenic medium (DMEM containing skeletal myogenic differentiating factor, 10 μM of 5-azacytidine) for 7 to 11 days, P-stem cells can fully differentiate into skeletal myocytcs;

2) culturing P-stem cells in cardiomyogenic medium [Iscove's Modified Dulbecco's Medium (IMDM) containing cardiomyogenic differentiating factor, 3 μM of 5-azacytidine) for 7 to 14 days, P-stem cells can fully differentiate into cardiomyocytes;

3) culturing P-stem cells in type-1 collagen pre-coated plate with renal cells induction medium [Embryo medium containing renal cell differentiating factor, 10 ng/ml of leukemia inhibitory factor (LIF)] for 21 to 28 days, P-stem cells can fully differentiate into renal cells;

4) culturing P-stem cells in pulmonary cell induction medium. [DMEM containing pulmonary cell differentiating factor, 10 μg/ml of insulin, 100 ng/ml of Fibroblast Growth Factor-1 (FGF-1), 200 ng/ml of FGF-2, 50 ng/ml of FGF-7, 800 ng/ml of FGF-9, 1,000 ng/ml of FGF-10, 1,000 ng/ml of FGF-18] for 14 to 21 days, P-stem cells can fully differentiate into pulmonary cells;

5) culturing P-stem cells in hepatogenic medium [low glucose-DMEM containing hepatogenic differentiating factor, 50 ng/ml of hepatocyte growth factor (HGF) and 100 ng/ml of FGF-4] for 14 to 21 days, P-stem cells can fully differentiate into hepatocytes;

6) culturing P-stem cells in adipogenic medium (DMEM containing 10% of fetal bovine serum and adipogenic differentiating factor, 1 μM of dexamethasone, 0.5 mM of methyl-isobutylxantine, 10 μg/ml of insulin, and 100 mM of indomethacin) for 72 hours and adipogenic medium with 10 μg/ml of insulin for additional 6 to 10 days, P-stem cells can fully differentiate into adipocytes. P-stem cells can be differentiated into any cell types in suitable induction media. Then, P-stem cell-derived target cells can repair the damaged tissue by directly transplanting them into the lesion.

### PRACTICE EXAMPLE 9

The constitutively expressed PKC isoforms in mononucleated cells are detected by Western Blot analysis with each PKC isoform-specific antibodies. FIG. 3 shows that mononucleated cells constitutively expressed PKC isoforms α, β1, β2, γ, l/λ and ζ. In the FIG. 1, Mo and pc represents mononucleated cell and PKC positive cell lysate, respectively. To examine the specific activation of PKC isoform(s) iii the differentiation of P-stem cells, mononucleated cells are pre-treated with Go6976 (1 μM) for 30 minutes at 37° C. and then incubated with Brvostatin-1 (10 nM) at designated time intervals. As shown in FIG. 4, only PKCβ2 is activated and translocates from cytosol to plasma membrane in the differentiation process of P-stem cells. Therefore, any substances stimulating the activation of PKCβ2 are capable of inducing the differentiation of P-stem cells.

### BRIEF DESCRIPTION OF THE DRAWING

FIG. 1A: The microscopic observation of monocytes (200× magnification).

FIG. 1B: The microscopic observation of P-stem cells (200× magnification).

FIG. 2A: The microscopic observation of Alizarin-stained osteoblasts (200× magnification).

FIG. 2B: The alkaline phosphatase activity of osteoblasts

FIG. 2C: The microscopic observation of polygonal chondrocytes (400× magnification).

FIG. 2D: The microscopic observation of Safranin O-stained osteoblasts (400× magnification).

FIG. 2E: The fluorescence microscopic observation of GAD-immunostained neuron cells (400× magnification).

FIG. 2F; The fluorescence microscopic observation of nestin-immunostained neuron cells (400× magnification).

FIG. 3: The determination of constitutively expressed PKC isoforms in monocytes by Western Blotting.

FIG. 4: The analysis of PKCβ2 translocation in Go6976/Bryostatin-treated monocytes.

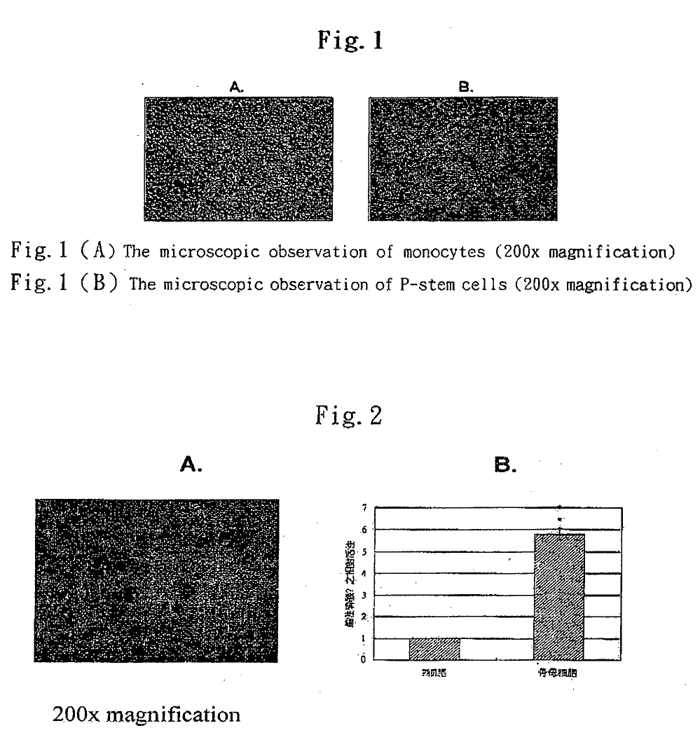

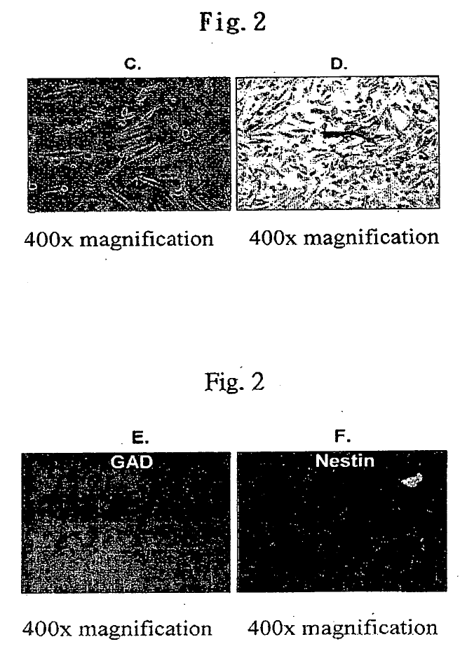

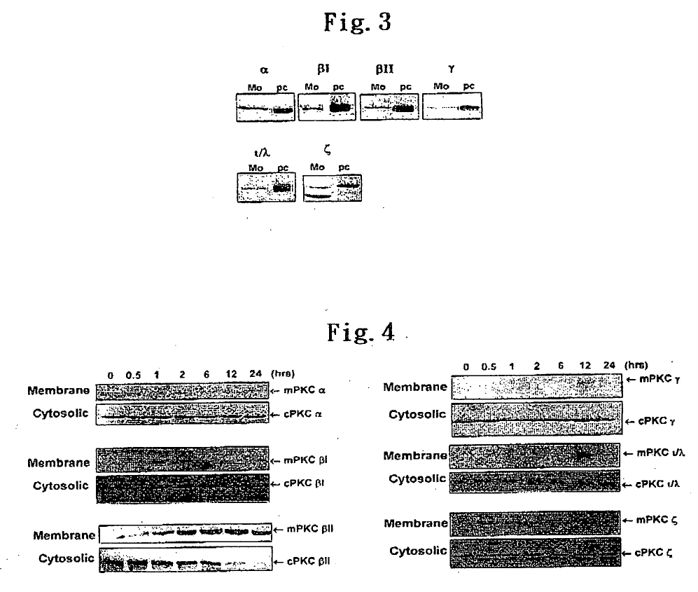

What is claimed is:

1. A P-stem cell is generated from one of mononucleated cells treated with protein kinase C (PKC) modulators.

2. A P-stem cell as claimed in claim 1, wherein the PKC modulator is Go6976, Bryostatin-1, or the combination of both.

3. A P-stem cell as claimed in claim 1, wherein the PKC modulator is GM-CSF, SDF or the combination of both.

4. A P-stem cell as claimed in claim 1, wherein the PKC modulator is collagen, fibronectin, or the combination of both.

5. A method of P-stem cell generation comprising the mononucleated cells differentiate into P-stem cells via activating intracellular PKCβ2.

6. A method of P-stem cell generation as claimed in claim 5, wherein the PKCβ2 activator is Go6976, Bryostatin-1, or the combination of both.

7. A method of P-stem cell generation as claimed in claim 5, wherein the PKCβ2 activator is GM-CSF, SDF or the combination of both.

8. A method of P-stem cell generation as claimed in claim 5, wherein the PKCβ2 activator is collagen, fibronectin, or the combination of both.

9. A target cell is differentiated from P-stem cells treated with differentiating factors and cultured in the induction media.

10. A target cell as claimed in claim 9, wherein the target cell is osteoblast; the osteogenic medium is low glucosc-DMEM; the osteogenic differentiating factors include dexamethasone, β-glyceropliosphate, ascorbic acid or other supplements.

11. A target cell as claimed in claim 9, wherein the target cell is chondrocyte; the chondrogenic medium is low glucose-DMEM; the chondrogenic differentiating factors include dexamethasone, TGF-β1, or other supplements.

12. A target cell as claimed in claim 9, wherein the target cell is neuron cell; the neurongenic medium is α-MEM; the neurongenic differentiating factors include mercaptoethanol, retinoic acid, L-glutamine, N2 supplement, B27 supplement, or other supplements.

13. A target cell as claimed in claim 9, wherein the target cell is cardiomyocyte; the cardiomyogenic medium is IMDM; the cardiomyogenic differentiating factors include 5-azacytidine or other supplements.

14. A target cell as claimed in claim 9, wherein the target cell is renal cell; the renal cell induction medium is Embryo medium; the renal cell differentiating factors include type-1 collagen, LIF or other supplements.

15. A target cell as claimed in claim 9, wherein the target cell is pulmonary cell; the pulmonary cell induction medium is DMEM; the pulmonary cell differentiating factors. include insulin, FGF-1, FGF-2, FGF-7, FGF-9, FGF-10, FGF-18, or other supplements.

16. A target cell as claimed in claim 9, wherein the target cell is hepatocyte; the hepatogenic medium is low glucose-DMEM; the, hepatogenic differentiating factors include HGF, FGF-4 or other supplements.

17. A target cell as claimed in claim 9, wherein the target cell is skeletal myocyte; the skeletal, myogenic medium is DMEM; the skeletal myogenic differentiating factors include 5-azacytidine or other supplements.

18. A target cell as claimed in claim 9, wherein the target cell is adipocyte; the adipogenic medium is DMEM containing 10% of fetal bovine serum; the adipogenic differentiating factors include dexamethasone, methyl-isobutylxantine, insulin, indomethacin, or other supplements.

19. A method of tissue repairing, comprising the tissue repairing by transplanting P-stem cells into the damaged tissues.

20. A method of tissue repairing, comprising the tissue repairing by transplanting target cells into the damaged tissue.
